(19) United States
(12) Patent Application Publication
(10) Pub. No.: US 2018/0327364 A1
(43) Pub. Date: Nov. 15, 2018

(54) MTOR INHIBITORS AND METHODS OF USE THEREOF

(71) Applicant: Brandeis University, Waltham, MA (US)

(72) Inventors: Ann Parrinello Lawson, Sudbury, MA (US); Jianjun Xue, Newton, MA (US); Lizbeth K. Hedstrom, Auburndale, MA (US); Rory T. Coffey, Brighton, MA (US)

(21) Appl. No.: 15/774,962

(22) PCT Filed: Nov. 14, 2016
(86) PCT No.: PCT/US16/61841

§ 371 Date: May 9, 2018

Related U.S. Application Data
(60) Provisional application No. 62/255,130, filed on Nov. 13, 2015.

(51) Int. Cl. C07D 239/49; C07C 279/24; C07C 271/20; C07C 279/30; A61P 35/00

(52) U.S. Cl. CPC C07D 239/49; C07C 279/24; A61P 35/00; C07C 279/30; C07C 271/20

(57) ABSTRACT

Disclosed are small molecule inhibitors of mammalian target of rapamycin (mTOR), and methods of using them.

### RELATED APPLICATION

This application claims the benefit of priority to U.S. Provisional Patent Application Ser. No. 62/255,130, filed Nov. 13, 2015. This application is hereby incorporated by reference in its entirety.

### GOVERNMENT SUPPORT

This invention was made with government support under R01-GM100921 awarded by the National Institutes of Health. The government has certain rights in the invention

### BACKGROUND

Mammalian target of rapamycin (mTOR) is an evolutionarily conserved serine/threonine kinase which serves as a master regulator of many cellular functions, including protein translation, autophagy, and cellular proliferation. mTOR integrates growth signals and the availability of amino acids. mTOR is found in two main complexes, mTOR complex 1 (mTORC1) and mTOR complex 2 (mTORC2), which phosphorylate different downstream targets. These two complexes are distinguished by the presence of Raptor in mTORC1 and Rictor in mTORC2. The insulin/Akt, MAPK/ERK, and Wnt pathways activate mTORC1. mTORC1 activates protein translation and inhibits autophagy. Insulin and growth factors stimulate mTORC2 via an unknown mechanism. mTORC2 controls cell survival and proliferation by phosphorylating Akt.

mTORC1 activates protein translation by phosphorylating both 4EBP1 and p70s6k. 4EBP1 inhibits formation of the eIF4F translation initiation complex that controls the translation of capped mRNAs. Phosphorylation disrupts the 4EBP1•eIF4E complex, allowing eIF4E to associate with eIF4F. mTORC1 is the only protein kinase known to phosphorylate 4EBP1. mTORC1 also phosphorylates p70s6k at Thr412, priming subsequent phosphorylation by PDK1 at Thr252 and by GSK3 at Ser371. Phosphorylation activates p70s6k, which in turn phosphorylates ribosome S6, activating translation of 5′ terminal oligopyrimidine tract (5′ TOP) mRNAs.

Dysregulation of mTOR1 is common in cancer, type 2 diabetes, and neurodegeneration. Additionally, the inhibition of mTOR1 prolongs lifespan in yeast, worms, fruit flies, and mice. Current inhibitors of mTOR generally fall within two categories: rapamycin (and rapamycin derivatives) and ATP-competitive mTOR kinase inhibitors. Rapamycin binds to mTORC1 as a complex with FKBP1. Rapamycin strongly inhibits phosphorylation of p70s6k at Thr389, but is much less effective at inhibiting 4EBP1 phosphorylation Thus, there exists a need for specific and selective inhibitors of mammalian target of rapamycin complex1 (mTORC1).

### SUMMARY

In certain embodiments, the invention relates to a compound having the structure of Formula (I), or pharmaceutically acceptable salt thereof

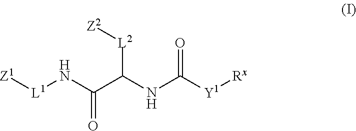

wherein:

Y¹ and Y² are independently NH, O, or S;

L¹ and L² are independently C₂-C₈ alkylene or C₂-C₈ alkenylene;

R^{x} is, independently for each occurrence, C₃-C₆ branched alkyl;

R^{y} is alkyl, aralkyl, cycloalkyl, or cycloalkylalkyl;

R^{z} is nitro or arylsulfonyl;

Z¹ is H, NH₂,

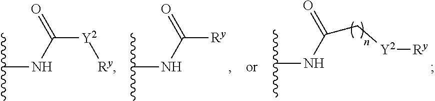

Z² is

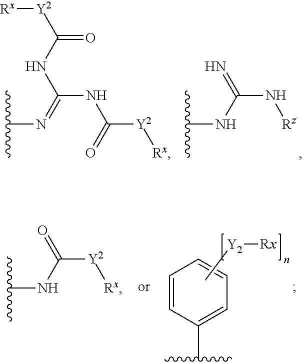

n is 1, 2, or 3: and

m is 1, 2, or 3.

In certain embodiments, the invention relates to any one of the compounds described herein, provided the compound is not a compound listed in Table 1, or an enantiomer thereof.

In certain embodiments, the invention relates to a pharmaceutical composition comprising any one of the aforementioned compounds and a pharmaceutically acceptable carrier.

In certain embodiments, the invention relates to a method of retarding the aging of a subject, comprising administering to the subject a therapeutically effective amount of any one of the aforementioned compounds or a compound listed in Table 1.

In certain embodiments, the invention relates to a method of preventing or treating diabetes in a subject, comprising administering to the subject a prophylactically or a therapeutically effective amount of any one of the aforementioned compounds or a compound listed in Table 1.

In certain embodiments, the invention relates to a method of preventing or treating a neurodegeneration or neurodegenerative disease in a subject in need thereof, comprising administering to the subject a prophylactically or a therapeutically effective amount of any one of the aforementioned compounds or a compound listed in Table 1.

In certain embodiments, the invention relates to a method of preventing or treating a cancer in a subject in need thereof, comprising the step of: administering to the subject a prophylactically or a therapeutically effective amount of any one of the aforementioned compounds or a compound listed in Table 1.

### BRIEF DESCRIPTION OF THE FIGURES

FIG. 1A-FIG. 1F depict data which show that B3A reduces luciferase protein. All samples were measured with Luciferase Assay System (Promega) on a luminometer and normalized with a Bradford assay. FIG. 1A is a bar graph showing that TMP-B3A reduces luciferase signal of both wild-type luciferase and luciferase-eDHFR fusion protein. The bar graph represents 5 independent replicates of cells treated with either DMSO or 200 TMP-B3A. FIG. 1B is a bar graph showing that B3A-containing ligands decrease luciferase signal. HEK-293T cells expressing HA-luciferase were treated for 4 h with 100 μM of the listed compounds. The bars represent the average and standard deviation of 4 independent replicates. FIG. 1C is a bar graph that shows that Cbz-B3A does not inhibit luciferase enzymatic activity. Recombinant luciferase was incubated with compound for 10 min then substrate was added and activity was measured. The bars represent the average and standard deviation of 5 independent replicates. FIG. 1D is an image of an immunoblot showing that Cbz-B3A reduces luciferase protein. HEK-293T cells expressing HA-luciferase were treated with 100 μM Cbz-B3A for 4 h. Cells were lysed and analyzed by immunoblotting with an anti-HA antibody. FIG. 1E is a bar graph representing 5 independent replicates of cells treated with either DMSO or 100 μM Cbz-B3A. FIG. 1F is a bar graph showing that Cbz-B3A reduces Renilla Luciferase. HEK-293T cells transfected with pRSV-Renilla were treated with 100 μM Cbz-B3A for 4 h. All samples were lysed in reporter lysis buffer and measured with substrate on a luminometer and normalized with a Bradford assay. The bars represent the average and standard deviation of 4 independent replicates. Significance determined in comparison to DMSO; n.s.: p>0.05, *: p<0.05, **: p<0.01, ***: p<0.001, ****: p<0.0001.

FIG. 2A-FIG. 2C depict data showing that Cbz-B3A inhibits protein translation. FIG. 2A is bar graph showing that Cbz-B3A does not induce degradation of luciferase. HEK-293T cells expressing HA-luciferase were pretreated with 100 μg/mL cycloheximide or 6 bortezomib for 15 min followed by treatment with 100 μM Cbz-B3A for 4 h. All samples were measured with Luciferase Assay System (Promega) on a luminometer and normalized by Bradford assay measurements. The bars represent average and standard deviation of 6 independent replicates. FIG. 2B is a pot showing that Cbz-B3A inhibits protein translation. HEK-293T cells were treated with vehicle or Cbz-B3A (300 nM, 1 μM, 3 μM, or 30 μM) for 4 H. Media was replaced with 35S labeled media containing the appropriate concentration of Cbz-B3A and cells were incubated for 1 h prior to lysis. Protein was isolated by TCA precipitation, washed, and 35S incorporation was measured on a scintillation counter. The graph represents the average and the standard deviation of 2 independent replicates. FIG. 2C is a bar graph showing that Cbz-B3A does not increase eIF2α phosphorylation. HEK-293T cells were treated with either Cbz-B3A (10 tunicamycin (5 μg/mL), thapsigargin (500 nM), or serum starved for 1 h. Bars represent the average and standard deviation of the quantification of 3 independent replicates analyzed by SDS-PAGE and immunoblotting.

FIG. 3A-FIG. 3C depict data showing that Cbz-B3A inhibits the phosphorylation of 4EBP1. FIG. 3A is an image of a immunoblot showing that Cbz-B3A reduces 4EBP1 phosphorylation. Specifically, an anti-4EBP1 western blot of lysate from HEK-293T cells treated with either DMSO, Cbz-Acetyl (10 Cbz-B3A (10 or rapamycin (20 nM) for 4 h is shown. FIG. 3B is a bar graph showing quantification of the Western blot (FIG. 3A) with hyper representing the left bar, mid the middle bar, and hypo the right bar. FIG. 3C is a plot showing dose response for inhibition of 4EBP1 phosphorylation. Cbz-B3A inhibits 4EBP1 phosphorylation with an EC50 of approximately 2 μM. The graph represents the quantification of 4EBP1 found in the top band (hyper-phosphorylation) and bottom band (hypo-phosphorylation) in a dilution curve of HEK-293T cells treated with Cbz-B3A for 4 h. Significance determined in comparison to DMSO; n.s.: p>0.05, *: p<0.05, **: p<0.01, ***: p<0.001, ****: p<0.0001.

FIG. 4A-FIG. 4C depict data showing that Cbz-B3A inhibits mTOR. FIG. 4A is a plot showing that Cbz-B3A inhibits p70S6K T389 phosphorylation. The graph represents the average and standard deviation of the quantification of anti T389 phosphorylation western blots done in three independent replicates. FIG. 4B is an image of an immunoblot showing that Cbz-B3A inhibits T389 but not S371 phosphorylation of p70s6k. p70s6k western blot of HEK-293T cells treated with either DMSO, Cbz-Acetyl (10 μM), Cbz-B3A (10 μM), or rapamycin (20 nM) for 4 h. Total anti-p70s6K and anti-phospho antibodies against T389 and S371 were used for blotting. FIG. 4C includes three bar graphs representing the quantification of the immunoblot in FIG. 4B. The bars represent the average and standard deviation of the western blots shown. No change observed in total p70s6k protein, though a reduction of phosphorylation explains the compactness of the band.

FIG. 5A-FIG. 5H depict data showing that Cbz-B3A and rapamycin have different downstream effects. FIG. 5A is an image of an immunoblot showing that Cbz-B3A has little to no effect on mTOR and mTOR phosphorylation levels. Representative anti-mTOR, p-mTOR S2448, and p-mTOR S2481 western blot of lysate from HEK-293T cells treated with DMSO, 10 μM Cbz-B3A, 20 nM rapamycin, or 10 μM of Cbz-Acetyl for 4 h. FIG. 5B is a bar graph showing that Cbz-B3A does not have a significant effect on mTOR S2448 phosphorylation. The bars represent the average and standard deviation of the quantification of the 3 independent replicates blotted in FIG. 5A. FIG. 5C is a bar graph showing that Cbz-B3A does not have a significant effect on mTOR levels. The bars represent the average and standard deviation of the quantification of the 3 independent replicates blotted in FIG. 5A. FIG. 5D is an image of an immunoblot showing that Cbz-B3A has no effect on Raptor levels. Anti-Raptor western blot of lysate from HEK-293T cells treated with DMSO, 10 μM Cbz-B3A, 20 nM rapamycin, or 10 μM of Cbz-Acetyl for 4 h. FIG. 5E is a plot showing that rapamycin inhibits translation. HEK-293T cells were treated with vehicle, 2 nM, 20 nM, and 200 nM rapamycin for 4 hours. After 4 h, media was replaced with 35S labeled media and cells were incubated for 1 hour then lysed. CPM from the lysate was measured and the graph represents average and standard deviation of 2 independent replicates. Experiment performed simultaneously with that of FIG. 2B. FIG. 5F is a bar graph showing that Cbz-B3A is a stronger inhibitor of translation than rapamycin. Cbz-B3A values are from FIG. 2B. Concentrations chosen based on the values of EC₅₀. FIG. 5G is a bar graph showing that Cbz-B3A increases autophagy. The bars represent the average and standard deviation of the quantification of 3 independent replicates blotted against LC3 II and treated as in FIG. 4A. FIG. 5H is a bar graph showing that Cbz-B3A has no effect on Akt phosphorylation. The bars represent the average and standard deviation of the quantification of 3 independent replicates blotted against phospho T308 and phospho S473 Akt and treated as in FIG. 4A. Significance determined in comparison to DMSO; n.s.: p>0.05, *: p<0.05, **: p<0.01.

FIG. 6A-FIG. 6H depict data showing that Cbz-B3A inhibits 4EBP1 phosphorylation through ubiquilins. FIG. 6A is an immunoblot showing that B3A binds to ubiquilin 1, ubiquilin 2, and ubiquilin 4. Anti-ubiquilin 1, 2, 4 western blots of acetyl and B3A pulldown. L: loading, F: flowthrough, W: 3rd wash, and Elut: elution. FIG. 6B is a plot showing that Cbz-B3A destabilizes ubiquilin 4. The graph represents the quantification of anti-ubiquilin 4 and actin western blots within a cellular thermal shift assay with treatment of 100 μM Cbz-B3A. Each data point represents the average and standard deviation of 2 independent replicates. FIG. 6C is an image of an immunoblot showing the ubiquilins knockdowns. Anti-ubiquilin western blots of samples to verify knockdown of specific ubiquilins. FIG. 6D are a series of bar graphs where the bars represent the average and standard deviation of the quantification of FIG. 6C. FIG. 6E is a bar graph showing that ubiquilin 2 and 4 affect mTORC1 activity. The bars represent the average and standard deviation of the quantification of 4EBP1 hypo-phosphorylation measured by western blots of HEK-293T cells treated with DMSO or 3 μM Cbz-B3A for 4 h. Cells were treated with either scramble, ubiquilin 1, ubiquilin 2, or ubiquilin 4 RNAi 72 h prior to Cbz-B3A treatment. Each experiment was done with 3 independent replicates. Protein knockdown verified in FIG. 6C and FIG. 6D. Significance of ubiquilin 2 RNAi DMSO and ubiquilin 4 RNAi 3 μM Cbz-B3A determined in comparison to scramble RNAi with the same treatment. FIG. 6F is a bar graph showing that the percent increase of hypo-phosphorylation from DMSO to 3 μM Cbz-B3A treatment calculated from FIG. 6E. Significance determined in comparison to DMSO. FIG. 6G is a bar graph showing that the effect of Cbz-B3A on Rheb. HEK-293T cells were treated as in FIG. 3A and immunoblotted against Rheb and actin. The bars represent the average and standard deviation of 3 independent replicates. The number above each bar represents the p value determined in comparison to DMSO. FIG. 6H is a bar graph showing the effect of Cbz-B3A on TSC2 as in FIG. 6G, and immunoblotted against TSC2 and actin. Significance found in comparison to DMSO; n.s.: p>0.05, *: p<0.05, **: p<0.01, ***: p<0.001.

FIG. 7A-FIG. 7C depict data showing that Cbz-B3A slows proliferation but is not cytotoxic. FIG. 7A is a plot showing the CellTiterGlo assay of K562, BaF3/p210, and HEK-293T cells treated for 48 h. Two different scales are used on the x-axis to visualize low concentrations. All data points represent the average and standard deviation of 3 independent replicates. FIG. 7B is a bar graph showing the LDH release assay of K562, BaF3/p210, and HEK-293T cells treated for 4 and 48 h. All data points represent the average and standard deviation of 3 independent replicates except K562 48 h which represents 2 independent replicates. FIG. 7C is a bar graph of cell lines showing lower than 50% proliferation from the NCI-60 DTP Human Tumor Cell Line Screen. Cells were treated for 48 h with 10 μM Cbz-B3A or 10 μM rapamycin. Rapamycin data was retrieved from NCI-60 DTP Human Tumor Cell Line Screen website.

FIG. 8 shows the NCI 60 cell screen data for Cbz-B3A. Cells were treated for 48 h with 10 μM Cbz-B3A. % proliferation was measured relative to untreated cells. First dash line represents average % proliferation of all cell lines. Second dash line represents 100% proliferation.

FIG. 9 is an image of two immunoblots showing that certain compounds of the invention reduce 4EBP1 phosphorylation. Specifically, an anti-4EBP1 western blot of lysate from HEK-293T cells treated with either DMSO, Cbz-B3A (20 μM), JX-3 (20 μM), JX-4 (20 μM), JX-5 (20 μM), JX-7 (20 μM), or JX-8 (20 μM), for 3 h is shown. Primary antibody of 1-1000× total 4EBP1 and secondary antibody of 1-5000× anti-rabbit were used. JX-8 appears to be a more potent mTOR inhibitor than CB3A.

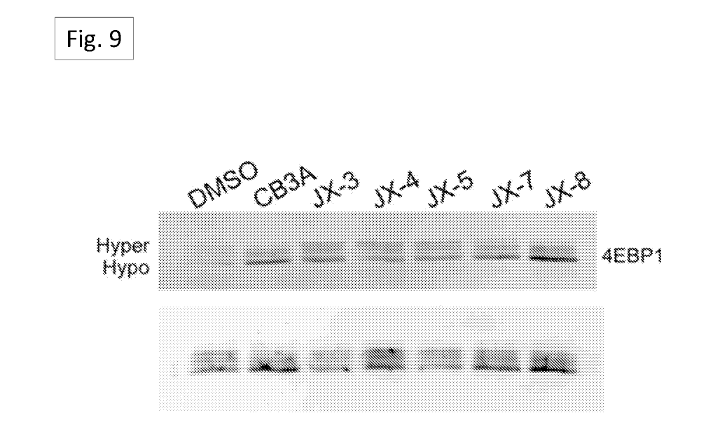

### DETAILED DESCRIPTION

### Overview

This invention is based at least in part on the unexpected discovered that certain compounds disclosed herein (e.g., B3A ligands) inhibit translation. For example, Cbz-B3A blocks translation by inhibiting the mTORC1 pathway in a process that is dependent on the presence of ubiquilins 2 and 4. Not wishing to be bound by theory but unlike other mTOR inhibitors, Cbz-B3A blocks the phosphorylation of 4EBP1 by mTORC1 and is a more effective inhibitor of translation than other mTORC1 inhibitors (e.g., rapamycin). The therapeutic potential of compounds of the invention to inhibit translation lends itself to effective treatment to prolong aging, for neurodegenerative disease, for diabetes, and for cancer (including as a combination therapy).

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulfonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom or ameliorates a condition according to clinically acceptable standards for the disorder or condition to be treated, e.g., at a reasonable benefit/risk ratio applicable to any medical treatment.

The term “prophylactic or therapeutic” is art-recognized and includes administration to the host of one or more of the subject compositions. If it is administered prior to clinical manifestation of the unwanted condition (e.g., disease or other unwanted state of the host animal) then it is prophylactic, (i.e., it protects the host against developing the unwanted condition), whereas if it is administered after manifestation of the unwanted condition, then it is therapeutic, (i.e., it is intended to diminish, ameliorate, or stabilize the existing unwanted condition or side effects thereof).

The term “patient” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer.

“Cycloalkyl” means mono- or multicyclic (e.g., bicyclic, tricyclic, etc.) or bridged saturated carbocyclic rings, each having from 3 to 12 carbon atoms. Likewise, preferred cycloalkyls have from 5-12 carbon atoms in their ring structure, and more preferably have 6-10 carbons in the ring structure.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “alkylthio” refers to an alkyl group, as defined above, having a sulfur moiety attached thereto. In certain embodiments, the “alkylthio” moiety is represented by one of —(S)-alkyl, —(S)-alkenyl, —(S)-alkynyl, and —(S)—(CH₂)ₘ—R¹, wherein m and R¹ are defined below. Representative alkylthio groups include methylthio, ethylthio, and the like.

The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R¹, where m and R¹ are described below.

The terms “amine” and “amino” are art-recognized and refer to both unsubstituted and substituted amines, e.g., a moiety that can be represented by the formulae:

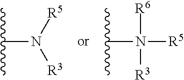

wherein R³, R⁵ and R⁶ each independently represent a hydrogen, an alkyl, an alkenyl, —(CH₂)ₘ—R¹, or R³ and R⁵ taken together with the N atom to which they are attached complete a heterocycle having from 4 to 8 atoms in the ring structure; R^{1R} represents an alkenyl, aryl, cycloalkyl, a cycloalkenyl, a heterocyclyl, or a polycyclyl; and m is zero or an integer in the range of 1 to 8. In certain embodiments, only one of R³ or R⁵ can be a carbonyl, e.g., R³, R⁵, and the nitrogen together do not form an imide. In even more certain embodiments, R³ and R⁵ (and optionally R⁶) each independently represent a hydrogen, an alkyl, an alkenyl, or —(CH₂)ₘ—R¹. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a conjugate acid with a pKₐ>7.00, i.e., the protonated forms of these functional groups have pKₐs relative to water above about 7.00.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 6- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like.

The term “arylsulfonyl” refers to an aryl group, as defined above, having a sulfonyl moiety attached thereto. Arylsulfonyl groups include, for example, benzenesulfonyl, p-toluenesulfonyl, 1-naphthalenesulfonyl, 2-naphthalelesulfonyl, and the like.

The term “carbonyl” is art-recognized and includes such moieties as can be represented by the formula:

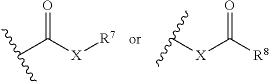

wherein X is a bond or represents an oxygen or a sulfur, and R⁷ represents a hydrogen, an alkyl, an alkenyl, —(CH₂)ₘ—R¹ or a pharmaceutically acceptable salt, R⁸ represents a hydrogen, an alkyl, an alkenyl or —(CH₂)ₘ—R¹, where m and R¹ are as defined above. Where X is an oxygen and R⁷ or R⁸ is not hydrogen, the formula represents an “ester.” Where X is an oxygen, and R⁷ is as defined above, the moiety is referred to herein as a carboxyl group, and particularly when R⁷ is a hydrogen, the formula represents a “carboxylic acid”. Where X is an oxygen, and R⁸ is a hydrogen, the formula represents a “formate.” In general, where the oxygen atom of the above formula is replaced by a sulfur, the formula represents a “thiocarbonyl” group. Where X is a sulfur and R⁷ or R⁸ is not hydrogen, the formula represents a “thioester” group. Where X is a sulfur and R⁷ is a hydrogen, the formula represents a “thiocarboxylic acid” group. Where X is a sulfur and R⁸ is a hydrogen, the formula represents a “thioformate” group. On the other hand, where X is a bond, and R⁷ is not hydrogen, the above formula represents a “ketone” group. Where X is a bond, and R⁷ is a hydrogen, the above formula represents an “aldehyde” group.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N₃; the term “cyano” means —CN; the term “isocyanato” means —NCO; the term “thiocyanato” means —SCN; the term “isothiocyanato” means —NCS; and the term “cyanato” means —OCN.

The phrase “protecting group”, as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2nd ed.; Wiley: New York, 1991). Protected forms of the inventive compounds are included within the scope of this invention. For example, “BOC-protected nitrogen,” “N—BOC,” and “BocHN” refer to a nitrogen atom to which a (CH₃)₃CO(O)C— is covalently bound. Similarly, “BOC-protected compound” refers to an organic compound that comprises a BOC-protected nitrogen.

As used herein, the definition of each expression, e.g., alkyl, m, n, etc., when it occurs more than once in any structure, is intended to be independent of its definition elsewhere in the same structure.

For purposes of this invention, the chemical elements are identified in accordance with the Periodic Table of the Elements, CAS version, Handbook of Chemistry and Physics, 67th Ed., 1986-87, inside cover.

### Exemplary Compounds

In certain embodiments, the invention relates to a compound having the structure of Formula (I), or pharmaceutically acceptable salt thereof

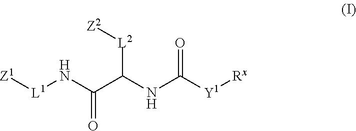

wherein:

Y¹ and Y² are independently NH, O, or S;

L¹ and L² are independently C₂-C₈ alkylene or C₂-C₈ alkenylene;

R^{x} is, independently for each occurrence, C₃-C₆ branched alkyl;

R^{y} is alkyl, aralkyl, cycloalkyl, or cycloalkylalkyl;

R^{z} is nitro or arylsulfonyl;

Z¹ is H, NH₂,

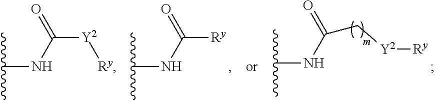

Z² is

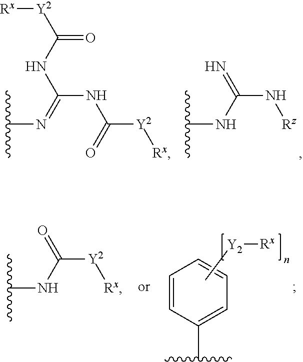

n is 1, 2, or 3; and

m is 1, 2, or 3.

In certain embodiments, the invention relates to any one of the compounds described herein, provided the compound is not a compound listed in Table 1, or an enantiomer thereof.

In certain embodiments, the invention relates to any one of the compounds described herein, wherein Y¹ is O.

In certain embodiments, the invention relates to any one of the compounds described herein, wherein L¹ is C₂-C₄ alkylene. In certain embodiments, the invention relates to any one of the compounds described herein, wherein L¹ is C₃ alkylene.

In certain embodiments, the invention relates to any one of the compounds described herein, wherein L² is C₂-C₆ alkylene. In certain embodiments, the invention relates to any one of the compounds described herein, wherein L² is C₆ alkylene.

In certain embodiments, the invention relates to any one of the compounds described herein, wherein Z¹ is

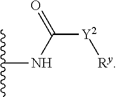

In certain embodiments, the invention relates to any one of the compounds described herein, wherein R^{y} is aralkyl. In certain embodiments, the invention relates to any one of the compounds described herein, wherein R^{y} is benzyl.

In certain embodiments, the invention relates to any one of the compounds described herein, wherein Z² is

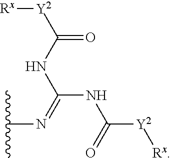

In certain embodiments, the invention relates to any one of the compounds described herein, wherein Z² is

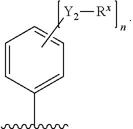

In certain embodiments, the invention relates to any one of the compounds described herein, wherein Z² is

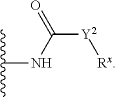

In certain embodiments, the invention relates to any one of the compounds described herein, wherein Y² is O.

In certain embodiments, the invention relates to any one of the compounds described herein, wherein R^{x} is C₃ or C₄ branched alkyl. In certain embodiments, the invention relates to any one of the compounds described herein, wherein R^{x} is C₄ branched alkyl. In certain embodiments, the invention relates to any one of the compounds described herein, wherein R^{x} is t-butoxycarbonyl.

In certain embodiments, the invention relates to any one of the aforementioned compounds, wherein the compound is a pharmaceutically acceptable salt.

### Exemplary Pharmaceutical Compositions

In certain embodiments, the invention relates to a pharmaceutical composition comprising any one of the aforementioned compounds and a pharmaceutically acceptable carrier.

Patients, including but not limited to humans, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

In certain embodiments, a dose of the compound will be in the range of about 0.1 to about 100 mg/kg, more generally, about 1 to 50 mg/kg, and, preferably, about 1 to about 20 mg/kg, of body weight of the recipient per day. The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3,000 mg, preferably 70 to 1400 mg of active ingredient per unit dosage form. An oral dosage of 50-1,000 mg is usually convenient.

Ideally, the active ingredient should be administered to achieve peak plasma concentrations of the active compound from about 0.2 to 70 μM, preferably about 1.0 to 15 μM. This can be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In certain embodiments, the mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose or sweetener as a sweetening agent and certain preservatives, dyes and colorings and flavors.

The compound or a pharmaceutically acceptable prodrug or salt thereof can also be mixed with other active materials that do not impair the desired action, or with materials that supplement the desired action, such as antibiotics, antifungals, anti-inflammatories or other antivirals, including but not limited to nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, carriers include physiological saline and phosphate buffered saline (PBS).

In certain embodiments, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

### Exemplary Methods

In certain embodiments, the invention relates to a method of retarding the aging of a subject, comprising administering to the subject a therapeutically effective amount of any one of the aforementioned compounds or a compound listed in Table 1.

In certain embodiments, the invention relates to a method of preventing or treating diabetes in a subject, comprising administering to the subject a prophylactically or a therapeutically effective amount of any one of the aforementioned compounds or a compound listed in Table 1. Diabetes includes, but is not limited to the following classes (or types): type I diabetes mellitus, type II diabetes mellitus, gestational diabetes, and other specific types of diabetes.

In certain embodiments, the invention relates to a method of preventing or treating a neurodegeneration or neurodegenerative disease in a subject in need thereof comprising, administering to the subject a prophylactically or a therapeutically effective amount of any one of the aforementioned compounds or a compound listed in Table 1. Neurodegenerative diseases includes but is not limited to Down syndrome, Alzheimer's disease, Parkinson's disease, Huntington's disease, Pick's disease, Gerstmann-Sträussler-Scheinker disease with tangles, amyotrophic-lateral sclerosis, AIDS-related dementia, fragile X-associated tremor/ataxia syndrome (FXTAS), progressive supranuclear palsy (PSP), and striatonigral degeneration (SND), which is included with olivopontocerebellear degeneration (OPCD) and Shy Drager syndrome (SDS) in a syndrome known as multiple syndrome atrophy (MSA), brain injury, amyotrophic lateral sclerosis and inflammatory pain, regenerative (recovery) treatment of CNS disorders such as spinal cord injury, acute neuronal injury (stroke, traumatic brain injury), guam-parkinsonism-dementia complex, corticobasal neurodegeneration, frontotemporal dementia, mood disorders.

In certain embodiments, the invention relates to a method of preventing or treating a cancer in a subject in need thereof, comprising the step of: administering to the subject a prophylactically or a therapeutically effective amount of any one of the aforementioned compounds or a compound listed in Table 1. In certain embodiments, the cancer is leukemia, myeloma, lung cancer (e.g., non-small cell lung cancer), colon cancer, central nervous system (CNS) cancer, melanoma, ovarian cancer, renal cancer, prostate cancer, or breast cancer.

In some embodiments, the method of treating cancer further comprises conjointly administering one or more additional chemotherapeutic agents.

Chemotherapeutic agents that may be conjointly administered with compounds of the invention include but are not limited to: ABT-263, aminoglutethimide, amsacrine, anastrozole, asparaginase, Bacillus Calmette-Guérin vaccine (bcg), bicalutamide, bleomycin, bortezomib, buserelin, busulfan, campothecin, capecitabine, carboplatin, carfilzomib, carmustine, chlorambucil, chloroquine, cisplatin, cladribine, clodronate, cobimetinib, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil and 5-fluorouracil, fluoxymesterone, flutamide, gemcitabine, genistein, goserelin, hydroxyurea, idarubicin, ifosfamide, imatinib, interferon, irinotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, nilutamide, nocodazole, octreotide, oxaliplatin, paclitaxel, pamidronate, pentostatin, pazopanib, perifosine, plicamycin, pomalidomide, porfimer, procarbazine, raltitrexed, rituximab, romidepsin, selumetinib, sorafenib, streptozocin, sunitinib, suramin, tamoxifen, temozolomide, temsirolimus, teniposide, testosterone, thalidomide, thioguanine, thiotepa, titanocene dichloride, topotecan, trametinib, trastuzumab, tretinoin, vinblastine, vincristine, vindesine, vinorelbine, and vorinostat (SAHA).

### EXEMPLIFICATION

The invention now being generally described, it will be more readily understood by reference to the following examples, which are included merely for purposes of illustration of certain aspects and embodiments of the present invention, and are not intended to limit the invention.

### Example 1—Compounds

TABLE 1

| Structure | Compound |
|---|---|
| 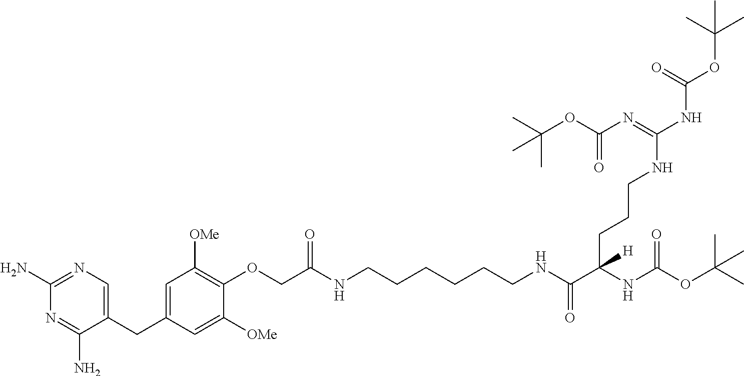 [SMILES: COc1cc(Cc2cnc(N)nc2N)cc(OC)c1OCC(=O)NCCCCCCNC(=O)[C@H](CCCN/C(=N\C(=O)OC(C)(C)C)NC(=O)OC(C)(C)C)NC(=O)OC(C)(C)C] | TMP-B3A |
| 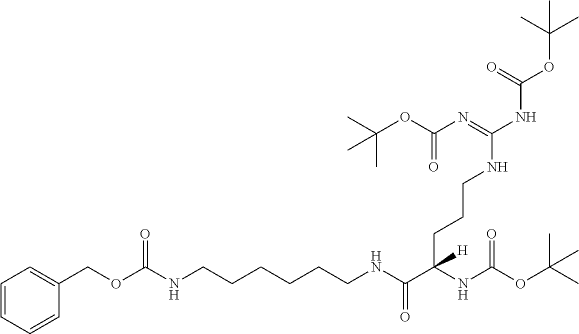 [SMILES: CC(C)(C)OC(=O)/N=C(\NCCC[C@H](NC(=O)OC(C)(C)C)C(=O)NCCCCCCNC(=O)OCc1ccccc1)NC(=O)OC(C)(C)C] | Cbz-B3A |
| 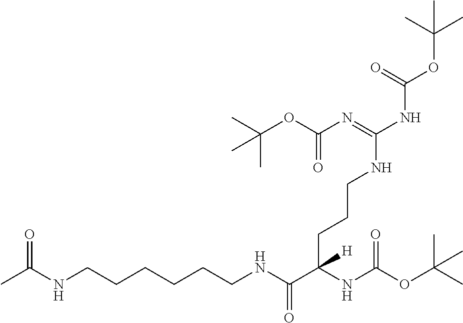 [SMILES: CC(=O)NCCCCCCNC(=O)[C@H](CCCN/C(=N\C(=O)OC(C)(C)C)NC(=O)OC(C)(C)C)NC(=O)OC(C)(C)C] | Acetyl- B3A |
| 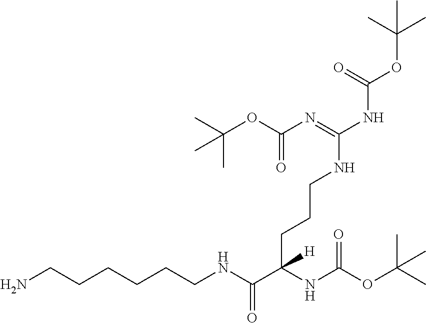 [SMILES: CC(C)(C)OC(=O)/N=C(\NCCC[C@H](NC(=O)OC(C)(C)C)C(=O)NCCCCCCN)NC(=O)OC(C)(C)C] | Amine- B3A |
| 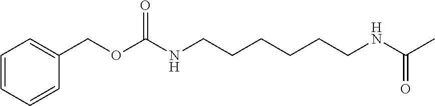 [SMILES: CC(=O)NCCCCCCNC(=O)OCc1ccccc1] | CBz- acetyl |
| 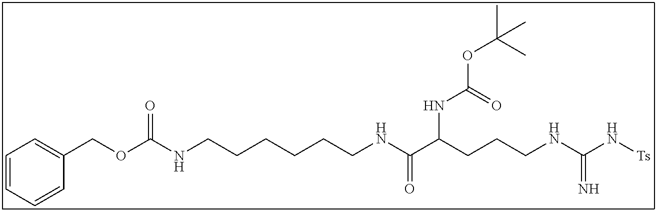 [SMILES: Cc1ccc(S(=O)(=O)NC(=N)NCCCC(NC(=O)OC(C)(C)C)C(=O)NCCCCCCNC(=O)OCc2ccccc2)cc1] | JX-3 |
| 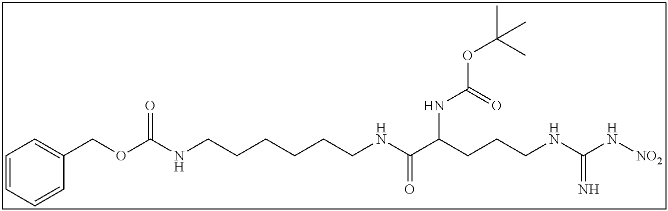 [SMILES: CC(C)(C)OC(=O)NC(CCCNC(=N)N[N+](=O)[O-])C(=O)NCCCCCCNC(=O)OCc1ccccc1] | JX-4 |
| 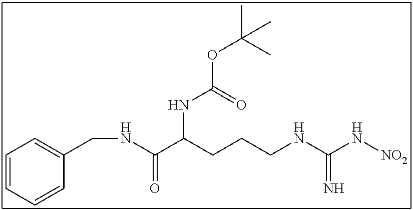 [SMILES: CC(C)(C)OC(=O)NC(CCCNC(=N)N[N+](=O)[O-])C(=O)NCc1ccccc1] | JX-5 |
| 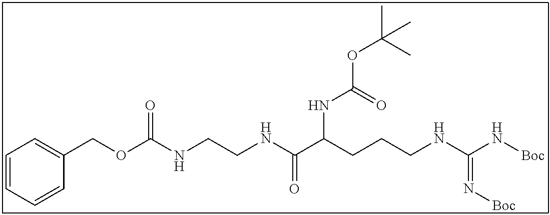 [SMILES: CC(C)(C)OC(=O)/N=C(/NCCCC(NC(=O)OC(C)(C)C)C(=O)NCCNC(=O)OCc1ccccc1)NC(=O)OC(C)(C)C] | JX-7 |
| 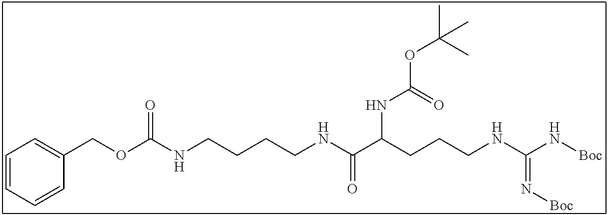 [SMILES: CC(C)(C)OC(=O)/N=C(/NCCCC(NC(=O)OC(C)(C)C)C(=O)NCCCCNC(=O)OCc1ccccc1)NC(=O)OC(C)(C)C] | JX-8 |

### Example 2—Experimental Procedures Pertaining to Example 3

Materials—

Rapamycin was obtained from Gold Biotechnology. Cycloheximide, tunicamycin, and thapsigargin were obtained from Sigma Aldrich. Bortezomib was obtained from Fisher Scientific. Rabbit monoclonal anti-p70^{s6k} (49D7, 2708), anti-p70^{s6k} T381 (108D2, 9234), anti-mTOR (7C10, 2983), anti-mTOR S2448 (D9C2, 5536), anti-mTOR S2481 (2974), anti-eIF2D S51 (D9G8, 3398), anti-LC3A/B (4108), anti-AKT S473 (D9E, 4060), anti-AKT T308 (D25E6, 13038), anti-Rheb (E1G1R, 13879), anti-TSC2 (D93F12, 4308), and rabbit polyclonal anti-4EBP1 (9452), and anti-p70^{s6k} S371 (9208) were obtained from Cell Signaling. Rabbit polyclonal anti-HA (ab9110), anti-raptor (ab40758), and anti-ubiquilin 4 (ab106443) and mouse monoclonal anti-ubiquilin 2 (ab57150) were obtained from Abcam. Mouse anti-Actin was obtained from Sigma. IRDye800CW donkey anti-mouse and IRDye680CW donkey anti-rabbit were obtained from Li-Cor.

Immunoblotting—

Quantification of western blots was performed with ImageJ. Cells were lysed in RIPA buffer (1% NP-40, 1% NaDeoxycholate, 0.1% SDS, 10 mM TrisCl, 10 mM β-glycerophosphate, 10 mM Na pyrophosphate, 50 mM NaF, 200 μM Na vanadate, 1 mM EDTA, 500 μM EGTA, 1% benzonase, and supplemented with complete (Roche Diagnostics) protease inhibitors).

Cell Culture and Experiments—

GP2-293 and HEK-293T cells were cultured in DMEM supplemented with 10% heat inactivated FBS (Sigma), 1× glutaMax (Gibco), 100 U/mL penicillin and 100 μg/mL streptomycin (Gibco) at 37° C. and 5% CO₂. K562 and BaF3/p210 cells were cultured in RPMI supplemented with 10% heat inactivated FBS (Sigma), 1× glutaMax, 100 U/mL penicillin and 100 μg/mL streptomycin at 37° C. and 5% CO₂.

Retroviral Transduction—

Firefly luciferase and firefly luciferase-eDHFR were cloned into pBABE-puro vector which was a gift from Hartmut Land & Jay Morgenstern & Bob Weinberg (Addgene plasmid #1764). pBABE-puro-luciferase was co-transfected with VSV-G into GP2-293 cells (Retro-X Universal Packaging System, Clonetech, Mountain View, Calif.) with TransIT-2020 (MirusBio) according to TransIT-2020's manufacture's protocol. After 48 h, viral supernatant was harvested, spun down, filtered with a 45 μM syringe filter, and added to HEK-293T supplemented with 8 μg/mL polybrene. After 4 h of infection, the media was replaced with fresh media. 48 h later, media was replaced with media containing 3 μg/mL puromycin. Media was replaced with fresh media containing puromycin every 3-4 days. After 2 weeks of puromycin selection, the cells were grown out from single cell colonies and screened for luciferase expression.

Luciferase Assay and Recombinant Luciferase—

Stably transduced luciferase HEK-293T cells were plated in 96-well plates 24 h prior to treatment with 100 μM of Cbz-B3A, amine-B3A, acetyl-B3A, and Cbz-acetyl. After 4 h, cells were harvested and measured with Luciferase Assay System with Reporter Lysis Buffer from Promega according to the manufacturer's protocol. Before the addition of luciferin, protein concentration was measured by removing 2 μl of lysate from each well and adding it to diluted Bio-Rad Protein Assay Dye Reagent Concentrate, and the absorbance was measured at OD₅₉₅. The protein concentration was used to normalize luciferase signal. For translation assays, cells were pretreated for 15 min with 100 μg/mL cycloheximide or 6 μM bortezomib. For the recombinant luciferase assay, Quantilum Recombinant Luciferase (Promega) was diluted 1:250,000 in reporter lysis buffer with 1 mg/mL BSA then mixed 1:1 with 200 μM compound in the same buffer for a final dilution of 1:500,000 and 100 μM compound in 20 μL. This was incubated for 10 min at room temperature then measured with Luciferase Assay System with Reporter Lysis Buffer from Promega according to the manufacturer's protocol.

Renilla Assay—

pRSV-Renilla was transfected into HEK-293T cells with TransIt 2020 according to manufacturer's protocol. Cells were trypsinized 48 h after transfection and plated into a 96 well plate at 10,000 cells/well. The media was removed 24 hours later and replaced by media containing 100 μM Cbz-B3A or DMSO and the cells were treated for 4 h. After treatment, cells were lysed with reporter lysis buffer (Promega), then substrate was added and renilla luciferase was measured on a luminometer. For the substrate, 10 μL of 10 mM coelenterazine (Gold Biotechnology) dissolved in acidified ethanol was added to 1 mL of renilla buffer (25 mM Na₄PPi, 10 mM NaOAc, 15 mM EDTA, 0.5 M Na₂SO₄, 1.0 M NaCl, pH 5.0).

³⁵S-Methionine/Cysteine Incorporation Translation Assay—

80,000 cells/well of HEK-293T cells were plated into 24 well plates 24 h before the assay. Cells were treated with indicated concentration of compound for 4 h. After 4 h, the media was removed and replaced with DMEM without methionine and cysteine (Lifetechnologies) supplemented with 0.2 mCi/mL EXPRESS [³⁵S] protein labeling mix (PerkinElmer), 10% dialyzed FBS (Pierce), 1× glutaMax (Gibco), 100 U/mL penicillin 100 μg/mL streptomycin (Gibco), 1 mM sodium pyruvate (Lifetechnologies), and the indicated concentration of compound. After 1 h, this media was removed, cells were washed with ice cold PBS, and lysed with low deoxycholate RIPA buffer (10 mM Tris-Cl, 1% NP-40, 0.5% Na Deoxycholate, 0.1% SDS, 140 mM NaCl, 1 mM EDTA, 500 μM EGTA, 1× Roche Complete protease inhibitors, 1:100 Benzonase, and at pH 8.0). Lysate was centrifuged at maximum speed for 20 m at 4° C. The concentration was measured by Bradford assay. 10 μg of lysate was added to 100 ice cold 1 mg/mL BSA with 0.02% Na Azide. 1 mL of ice cold 10% TCA was added, the solutions were mixed and incubated on ice for 0.5 h. These mixtures were vacuum filtered onto GF/C Whatman glass microfiber filters. The filters were washed with 2×5 mL of 10% TCA, then 2×3 mL ethanol. The filters were then dried for a 0.5 h. The filters were placed into scintillation vials with 5 mL of scintillation fluid and read on a scintillation counter.

SILAC Lysate—

HEK-293T cells were grown in DMEM media minus L-lysine and L-arginine (Thermo Scientific Pierce) supplemented with 10% dialyzed FBS for SILAC (Pierce), 1× glutaMax (Gibco), 100 U/mL penicillin 100 μg/mL streptomycin (Gibco) and either 84 μg/mL [¹³C/¹⁵N]-L-arginine and [¹³C/¹⁵N]146 μg/mL L-lysine (Cambridge Isotope Laboratories) or 84 μg/mL L-arginine and 146 μg/mL L-lysine (Sigma) at 37° C. and 5% CO₂ for a minimum of 6 passages. Cells were lysed by 3 quick freeze/thaws in DPBS supplemented with Roche complete protease inhibitor cocktail and 10 mM β-glycerophosphate, 10 mM Na pyrophosphate, 50 mM NaF, and 200 μM Na vanadate and used for pulldowns. Mass spectrometry was done by a Thermo Orbitrap Elite and Thermo XL-ETD Orbitrap microcapillary LC-MS/MS.

Pulldowns—

NHS-activated Sepharose 4 fast flow beads (GE Healthcare Life Sciences) were added to screw cap spin columns (Pierce) washed with phosphate buffer. Amine-acetyl or amine-B3A was dissolved in ethanol at 5 mM then diluted 5-fold into 100 mM NaPO₄, 150 mM NaCl at pH 7.2 to reach a concentration of 1 mM of compound in buffer. 1 mM amine-acetyl or amine-B3A was added to beads and rotated overnight at room temperature. Afterwards, the beads were blocked with 100 mM ethanolamine for 3 h. Manufacturer's protocol was followed thereafter. Briefly, HEK-293T cells were lysed by 3 quick freeze/thaws in DPBS supplemented with Roche complete protease inhibitor cocktail and 10 mM β-glycerophosphate, 10 mM Na pyrophosphate, 50 mM NaF, and 200 μM Na vanadate. Lysate was added to the beads at 3 μg/μl and rotated for 30 min at room temperature, then washed 3 times with DPBS and inhibitors, then eluted with 1× Laemmli loading buffer at 65° C. for 20 min. For mTOR pulldowns, HEK-293T cells were grown, washed twice with PBS, and lysed with lysis/wash buffer (40 mM HEPES pH 7.0, 120 mM NaCl, 0.3% CHAPS, 10 mM NaF, 10 mM glycerophosphate, 10 mM Na pyrophosphate, 10 mM Na azide, 200 μM Na vanadate, and 1× Roche complete protease inhibitor). Lysate was precleared with protein G magnetic beads (Pierce) for 1 H at 4° C., beads were removed from the lysate, then mTOR antibody (Santa Cruz, N-19) was added and rotated for 3 H at 4° C. followed by the addition of protein G beads, which was then rotated at 4° C. overnight. Beads were washed 3 times with lysis/wash buffer then eluted with 1× Laemmli buffer at 100° C. for 10 minutes.

Cellular Thermal Shift Assay (CETSA)—

CETSA was performed as previously described. Briefly, HEK-293T cells were lysed in DPBS with Roche complete protease inhibitor by 3 quick freeze thaws, centrifuged at 20,000 g for 20 min at 4° C., and the supernatant was adjusted to 4 mg/mL. Lysate was incubated with compound for 30 min and heated to different temperatures in a thermal cycler for 3 min, then cooled to room temperature for 3 min. All samples were then centrifuged at 20,000 g for 20 min at 4° C. and a western blot was run on the supernatant.

RNAi Knockdown—

ON-TARGETplus SMARTpool siRNA for ubiquilin 1, ubiquilin2, and ubiquilin 4 was obtained from Dharmacon and transfected into HEK-293T cells with DharmaFECT 1 (Dharmacon, Lafayette, Colo.) according to manufacturer's protocol. Cells were harvested 72 h post transfection and analyzed by western blot.

Cellular Growth and Cytoxicity Assays—

To determine cellular growth, CellTiter-Glo luminescent cell viability assay (Promega) was used per the manufacturer's instruction. To determine cytotoxicity, LDH release was measured with the LDH Cytotoxicity Assay Kit (Pierce) according to the manufacture's protocol. For 4 h assays, 96 well plates were seeded at 75,000 cells/mL for BaF3/p210 cells or K562 cells. For HEK-293T cells, 10,000 cells/well were plated 24 h before treatment. Cells were treated for 4 h at 37° C. and 50 was removed from every well and used in the LDH Cytotoxicity assay per the manufacturer's protocol. For 48 h assays, 7,500 cells/mL of BaF3/p210 or K562 cells were incubated with compound at 37° C. for 48 h (200 μL/well). For HEK-293T cells, 1,000 cells/well were plated 24 h prior to compound treatment. After treatment, 50 μL was removed from each well for the LDH assay for all cell lines and 100 μL of mixed cells were removed and used with the CellTiter-Glo kit for K562 and BaF3/p210 cells. For HEK-293T cells, wells were treated directed with the CellTiter-Glo kit. Cytotoxicity or viability was measured by luminosity and absorbance on a microplate reader.

NCI-60 DTP Human Tumor Cell Line Screen—

Screen was conducted as previously described, performed at Developmental Therapeutics Program NCI/NIH.

Statistics—

All p-values were determined by a standard independent 2 sample t-test calculated by scipy.stats.ttest_ind within Python 3.4 (programming language; www.python.org)

### Example 3—Cbz-B3A Inhibits mTORC1 Signalling in Cells

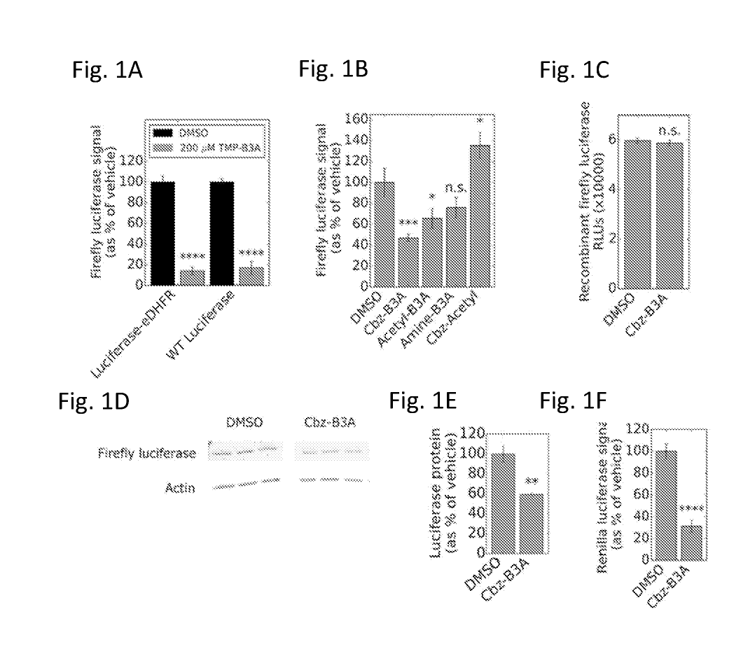

Cbz-B3A decreases luciferase protein. To investigate the mechanism of B3A induced degradation, HEK-293T cells were constructed that stably expressed firefly luciferase fused to eDHFR (luciferase-eDHFR), as well as firefly luciferase alone (wild type) as a control. Surprisingly, TMP-B3A reduced luciferase signal to similar levels in both luciferase-eDHFR and wild-type luciferase cells (FIG. 1A). Firefly luciferase is a promiscuous small molecule binder, suggesting that TMP-B3A might bind to luciferase and induce proteasomal degradation as previously reported for the B3A-induced degradation of eDHFR and GST. Therefore the TMP recognition ligand was removed to leave the free amino group at the end of the linker (amine-B3A). Because the positive charge of the free amine was likely to decrease cellular uptake relative to TMP-B3A, TMP was replaced with amine protecting groups carboxybenzyl and acetyl to make Cbz-B3A and acetyl-B3A, respectively (see Table 1). Amine-B3A, Cbz-B3A and acetyl-B3A also decreased the wild type luciferase signal (FIG. 1B). In contrast, the luciferase signal was not reduced when cells were treated with Cbz-acetyl, which lacks the B3A tag. These observations indicate the B3A tag is responsible for the decrease in firefly luciferase signal, although the process is distinct from the B3A-induced proteasomal degradation observed previously.

Next, whether the reduction in signal was due to inhibition of luciferase activity or reduced levels of luciferase protein was determined. When recombinant firefly luciferase was incubated with Cbz-B3A, there was no reduction of signal, indicating that Cbz-B3A does not inhibit the enzymatic activity of firefly luciferase (FIG. 1C). However, when cells were treated with Cbz-B3A, the amount of luciferase protein decreased by approximately 40%, which is in good agreement with the decrease in luciferase signal (See for example, FIGS. 1B, 1D and 1E). To confirm that this decrease was not specific to the MoMuLV LTR promoter or an anomaly with firefly luciferase, the assay with HEK-293T cells transfected with renilla luciferase regulated by the RSV promoter was repeated. A similar decrease in signal was observed with Cbz-B3A treatment (FIG. 1F). Thus Cbz-B3A decreased the levels of both luciferase proteins.

The Cbz-B3A Mediated Decrease in Luciferase Protein is not Proteasome Dependent.

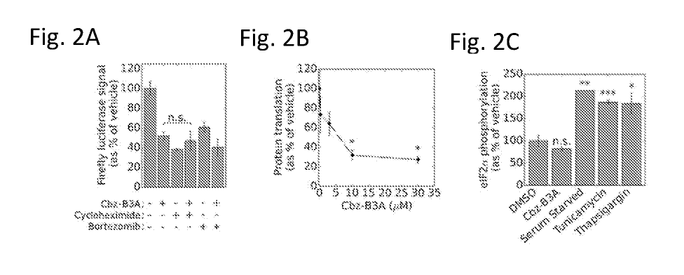

To further investigate why luciferase protein levels decrease with Cbz-B3A treatment, cells were treated with bortezomib to inhibit proteasomal degradation. Bortezomib alone decreased luciferase levels (FIG. 2A). Similar decreases in luciferase levels have been reported by others when cells are treated with proteasome inhibitors. Firefly luciferase signal decreased when cells were co-treated with Cbz-B3A and bortezomib when compared to bortezomib alone. These observations indicate that Cbz-B3A does not decrease firefly luciferase through proteasomal degradation (FIG. 2A).

Cbz-B3A Inhibits Translation—

Since the Cbz-B3A-induced decrease of luciferase is not proteasome dependent, whether Cbz-B3A inhibits translation was investigated. When firefly luciferase expressing cells were treated with the translation inhibitor cycloheximide, luciferase activity decreased as expected (FIG. 2A). When these cells were co-treated with cycloheximide and Cbz-B3A, no further decrease in luciferase activity was observed (FIG. 2A), suggesting that Cbz-B3A inhibits translation of luciferase. The incorporation of ³⁵S-methionine/cysteine into proteins was measured to directly determine the effects of Cbz-B3A on global translation. Cbz-B3A decreased the incorporation of ³⁵S-methionine/cysteine into protein in a dose dependent manner (FIG. 2B), with maximal inhibition of 68% observed at 10 μM, and an EC₅₀ of approximately 3 μM. Thus, Cbz-B3A is a strong inhibitor of translation.

Cbz B3A does not Inactivate eIF2α—

B3A resembles an unfolded peptide, which suggests that it might trigger the unfolded protein response (UPR). Translation is blocked by the phosphorylation of the translation initiation factor eIF2α during UPR, as well as during ER stress, and in response to amino acid starvation. Therefore, whether B3A induced the phosphorylation of eIF2α was investigated. As expected, serum starvation, tunicamycin (induces ER stress and UPR), and thapsigargin (induces ER stress and UPR) increased phosphorylation of eIF2α (FIG. 2C). However, there was no increase in eIF2α phosphorylation after treatment with Cbz-B3A when compared with vehicle. Thus Cbz-B3A does not block translation by inducing the phosphorylation of eIF2a.

Cbz-B3A Inhibits the Phosphorylation of 4EBP1—

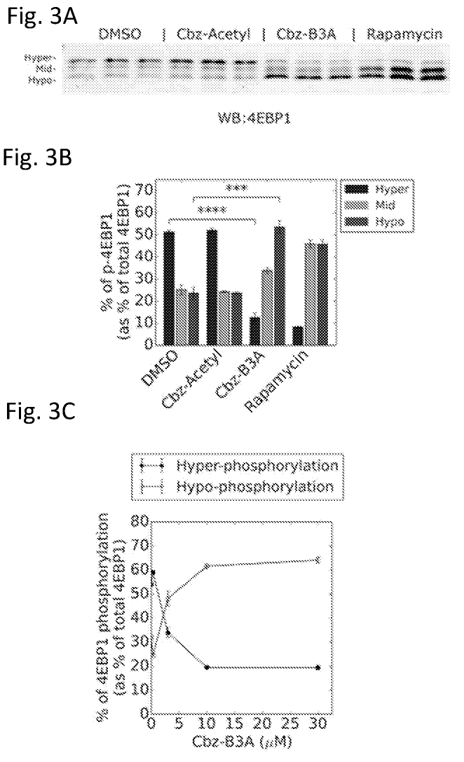

The effect of Cbz-B3A on the translation repressor 4EBP1 was investigated. The phosphorylation of 4EBP1 by mTORC1 inactivates 4EBP1, allowing translation to initiate. 4EBP1 has multiple phosphorylation sites, the differentiation of which is visible on a western blot of total 4EBP1 protein (FIG. 3A). The top 4EBP1 band is the translation-on hyper-phosphorylated form and the bottom band is the translation-off hypo-phosphorylated form. When cells were treated with 10 μM Cbz-B3A, a clear shift from hyper-phosphorylation to hypo-phosphorylation was observed (FIGS. 3A and 3B). No shift was observed with Cbz-acetyl, which indicates that the B3A tag is required for the inhibition of 4EBP1 phosphorylation. The dose response of 4EBP1 phosphorylation inhibition reached a maximum at 10 μM with an EC₅₀ of approximately 2 in good agreement with the dose dependence of translation inhibition (compare FIG. 3C and FIG. 2B). Moreover, the accumulation of hypo-phosphorylation was greater than that observed with saturating rapamycin (FIGS. 3A and 3B). The incomplete inhibition of 4EBP1 phosphorylation by rapamycin is consistent with previous reports. These observations indicate that Cbz-B3A blocks translation by inhibiting the phosphorylation of 4EBP1. Cbz-B3A Inhibits mTORC1 Signaling—

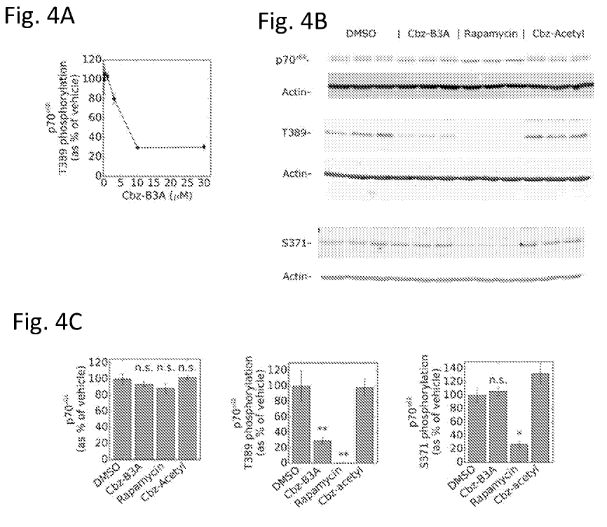

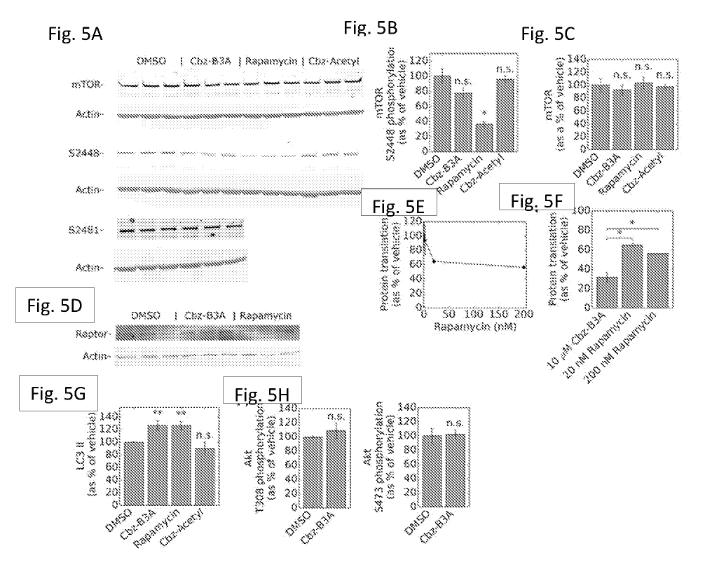

The phosphorylation of p70^{s6k} was measured to further investigate the effects of Cbz-B3A on mTORC1 regulated translation. Cbz-B3A inhibits phosphorylation of p70^{s6k} at Thr389 (FIGS. 4A and 4B). Maximum inhibition was reached at 10 with an EC₅₀ of approximately 5 as observed with 4EBP1 (FIG. 4A). Interestingly, Cbz-B3A treated cells retained approximately 30% phosphorylation at Thr389, while rapamycin completely blocked phosphorylation of this site. Unlike rapamycin, Cbz-B3A did not inhibit phosphorylation of p70^{s6k} at Ser371 (FIGS. 4B and 4C). The p70^{s6k} catalyzed phosphorylation of mTOR at Ser2448 was also examined. Cbz-B3A had only a small effect on the phosphorylation of mTOR at Ser2448 (FIGS. 5A and 5B), indicating that Cbz-B3A mediated decrease in the phosphorylation of Thr389 does not inactivate p70^{s6k}. Importantly, the levels of mTOR did not change after treatment with Cbz-B3A (FIG. 5C), nor did Raptor levels (FIG. 5D).

Recent literature indicates that phosphorylation of 4EBP1 is more important than the phosphorylation of p70^{s6k} in regulating the rate of protein translation, suggesting that Cbz-B3A should be a more effective translation inhibitor than rapamycin. Indeed, whereas saturating concentrations of Cbz-B3A inhibited translation by 68%, rapamycin inhibited translation by only 35% (FIGS. 5E and 5F).

The inhibition of mTORC1 activates autophagy, and the amount of autophagy correlates with the amount of LC3 A/B II. Cbz-B3A increased LC3 A/B II to equivalent levels as rapamycin (FIG. 5G), indicating that both compounds increase autophagy. This observation is further evidence that Cbz-B3A inhibits mTORC1 signaling.

Cbz-B3A does not Inhibit mTORC2—

Previously reported inhibitors of mTORC1 also inhibit mTORC2, so the effects of Cbz-B3A on the mTORC2 catalyzed phosphorylation of Akt at Ser473 were examined. No decrease in Akt phosphorylation was observed when cells were treated with Cbz-B3A, indicating that Cbz-B3A only inhibits the mTORC1 complex (FIG. 5H). Akt is also upstream of mTORC1, so this observation also indicates Akt does not mediate the inhibition of mTORC1 by Cbz-B3A.

Phosphorylation of mTOR at Ser2481 is a marker of the mTORC2 complex. There was no change in Ser2481 phosphorylation of mTOR after treatment with Cbz-B3A indicating that there was no shift of mTOR from mTORC1 to mTORC2 (FIG. 5A). Thus Cbz-B3A does not decrease the levels of mTOR and Raptor, nor does it change the ratio of mTORC1 and mTORC2.

Cbz-B3A Binds Ubiquilins—

A stable isotope labeling of amino acids in cell culture (SILAC) bead pulldown assay was implemented to identify proteins that bind the B3A tag. HEK-293T cells were incubated with either media supplemented with [¹³C/¹⁵N]-L-arginine and [¹³C/¹⁵N]-L-lysine (R6K6) or unlabeled L-arginine and L-lysine (R0K0). B3A beads were incubated with R6K6 lysate and acetyl beads were incubated with R0K0 lysate for a negative control. Proteins were eluted with 1× Laemmli buffer and the eluent was analyzed by mass spectrometry. mTOR and the other components of mTORC1 (Raptor, mLST8, PRAS40, and DEPTOR) were not found in this sample, indicating that mTORC1 does not bind Cbz-B3A directly. One hundred forty six proteins were identified with a B3A to acetyl ratio of greater than 2:1, including 47 proteins that were only found in the B3A sample. Ubiquilin 2 was at the top of this list, with seven peptides accounting for 19.9% sequence coverage, including 3 unique peptides. Two homologs of ubiquilin 2, ubiquilin 1 and ubiquilin 4, also bind selectively to the B3A resin, with B3A to acetyl ratios of 7.3:1 and 2.7:1, respectively. Intriguingly, ubiquilin 1 has been reported to bind to mTOR, though the effects of this interaction are unknown.

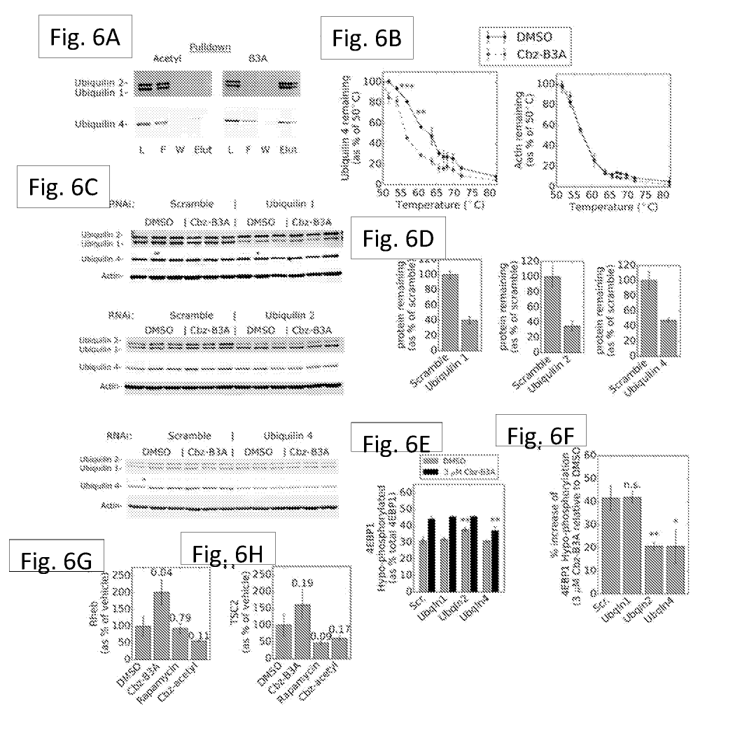

Ubiquilins bind to B3A with immunoblotting. B3A beads bound ubiquilin 1 and ubiquilin 2 sufficiently to deplete the lysate (FIG. 6A). Ubiquilin 4 bound and eluted from B3A beads but did not appear to bind as strongly as ubiquilin 1 and 2. In contrast, no ubiquilin eluted from acetyl beads. mTOR was present in both B3A and acetyl eluants, demonstrating that this interaction was not specific and further indicating that B3A does not bind directly to mTOR.

To further confirm Cbz-B3A interacts with ubiquilins, cellular thermal shift assays (CETSA) were performed. In CETSA, the thermal stability of a protein is monitored by incubating lysate at different temperatures. When the protein of interest denatures, it precipitates and is depleted from the soluble fraction. Ligands usually stabilize a protein and are expected to shift the melting curve to higher temperatures, although shifts to lower melting points have also been observed. When lysate was incubated with Cbz-B3A, there was a clear shift to a lower melting temperature for ubiquilin 4 (FIG. 6B), indicating Cbz-B3A destabilizes this protein. No shift was seen in actin, demonstrating that this was a specific effect. This observation suggests that Cbz-B3A binds to ubiquilin and induces a conformational change. Unfortunately, neither ubiquilin 1 nor ubiquilin 2 melted within the accessible temperature range.

CBz-B3A Inhibits mTOR Through Ubiquilins—

In order to address whether the association of Cbz-B3A with ubiquilins mediates the inhibition of mTORC1, RNAi knockdowns of ubiquilin 1, 2, and 4 were performed. Ubiquilin 1, 2, and 4 were knocked down 60%, 65%, and 52% respectively (FIGS. 6C and 6D). No off target or compensating effects were observed on other ubiquilins. Ubiquilin 2 RNAi increased the hypophosphorylated form of 4EBP1 (FIG. 6E), suggesting that ubiquilin 2 activates mTORC1. No change in 4EBP1 phosphorylation was observed with either ubiquilin 1 or ubiquilin 4 knockdown.

The effect of ubiquilin knockdown in the context of Cbz-B3A treatment was also observed. Cells were treated with 3 μM Cbz-B3A so that either an increase or a decrease in 4EBP1 phosphorylation could be detected. The knockdown of both ubiquilin 2 and ubiquilin 4 decreased the ability of Cbz-B3A to block the phosphorylation of 4EBP1, while the knockdown of ubiquilin 1 had no effect on Cbz-B3A (FIGS. 6E and 6F). Thus ubiquilin 2 and 4 mediate the inhibition of mTORC1 by Cbz-B3A.

Ubiquilins 2 and 4 do not Interact with mTOR—

FRET assays were performed to determine whether ubiquilin binds to mTOR within the context of living cells. HEK-293T cells were transfected with ubiquilin 1, 2, or 4 fused to Cerulean as the donor and mTOR fused with Venus as the acceptor. Both the N-terminal and C-terminal fusion proteins were tested. FRET efficiency was measured by FRET after acceptor photobleaching. No evidence of FRET was observed with any of the combinations of ubiquilin and mTOR in the absence or presence of Cbz-B3A.

Cbz B3A May Increase the Levels of TSC2 and Rheb—

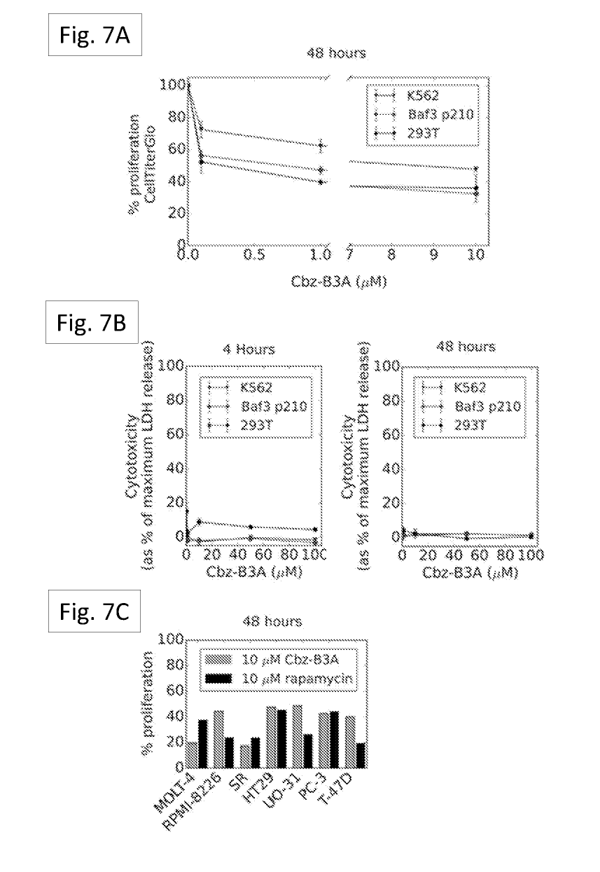

Ubiquilins modulate protein degradation, increasing the degradation of some proteins and protecting others. However, no change in the levels of mTOR, 4EBP1, p70^{s6k}, or Raptor was observed with Cbz-B3A treatment (see FIG. 3A, FIG. 4B, FIG. 5A and FIG. 5D). Two proteins that modulate mTORC1 activity were also enriched in the B3A eluant, Ras homolog enriched in brain (Rheb) and its GTPase activating protein TSC2. Rheb is a direct upstream activator of mTORC1 while TSC2 inhibits Rheb action. Rheb was ranked 109 with a B3A:acetyl ratio of 2.5:1. TSC2 ranked 22^{nd} and was only found in the B3A eluant. Cbz-B3A treatment increased the amount of Rheb (FIG. 6G; p=0.04). Cbz-B3A also appeared to increase the amount of TSC2, although this increase did not reach the level of 95% confidence (FIG. 7H; p=0.19).

Cbz-B3A Slows Cellular Growth—

Since inhibition of mTORC1 slows cellular proliferation, the proliferation of several different cell lines after treatment with Cbz-B3A was investigated. The proliferation of HEK-293T, K562, and BaF3/p210 cells, measured by CellTiter Glo, was significantly slowed by treatment with as little as 1 μM Cbz-B3A for 48 h. Maximum growth inhibition was seen at 10 μM Cbz-B3A treatment. Proliferation of HEK-293T, K562, and BaF3/p210 was inhibited by 64%, 52%, and 68%, respectively (FIG. 7A). Importantly, Cbz-B3A was not cytotoxic to the cells as measured by the release of lactate dehydrogenase (FIG. 7B).

### Example 4—Effect of Cbz-B3A on Proliferation in the NCI 60 Screen

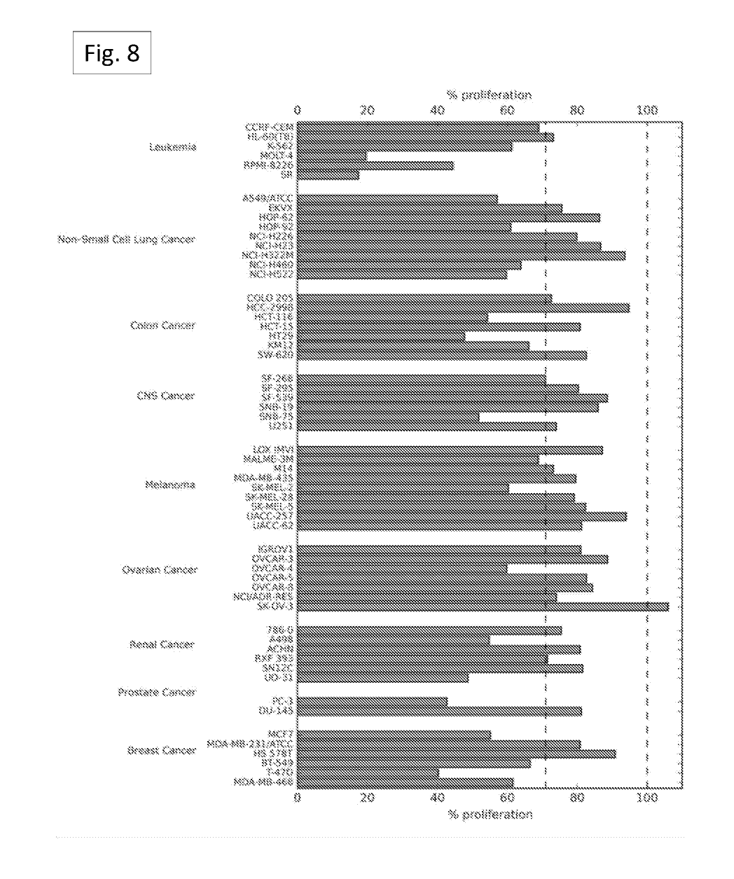

Cbz-B3A was also evaluated in the NCI-60 DTP Human Tumor Cell Line Screen. The average decrease in proliferation was 29% for all cell lines after 48 h treatment with 10 μM Cbz-B3A. The inhibition of proliferation for K562 cells was 39%, consistent with CellTiter Glo findings (FIG. 8). The most sensitive cell lines were MOLT-4 and SR with a decrease in proliferation of 80% and 83% respectively (FIG. 7C). MOLT-4 and SR cell lines were also more sensitive to 10 μM Cbz-B3A than 10 μM rapamycin. Both MOLT-4 and SR cell lines derive from leukemia patients and Cbz-B3A has the largest effect within this category of cancer. See FIG. 8.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (I) or a pharmaceutically acceptable salt thereof: 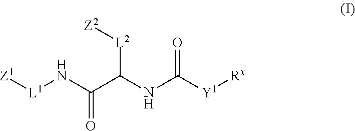
wherein:
Y¹ and Y² are independently NH, O, or S;
L¹ and L² are independently C₂-C₈ alkylene or C₂-C₈ alkenylene;
R^{x} is, independently for each occurrence, C₃-C₆ branched alkyl;
R^{y} is alkyl, aralkyl, cycloalkyl, or cycloalkylalkyl;
R^{z} is nitro or arylsulfonyl;
Z¹ is —H, NH₂, 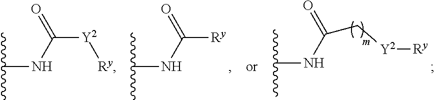
Z² is 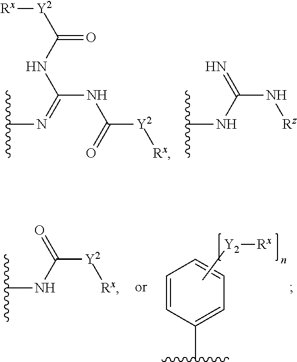
n is 1, 2, or 3:
m is 1, 2, or 3.

2. The compound of claim 1, provided the compound is not a compound listed in Table 1, or an enantiomer thereof.

3. The compound of claim 1, wherein Y¹ is O.

4. The compound of claim 1, wherein L¹ is C₃ alkylene.

5. The compound of claim 1, wherein L² is C₆ alkylene.

6. The compound of claim 1, wherein Z¹ is

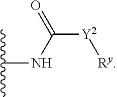

7. The compound of claim 1, wherein Z² is

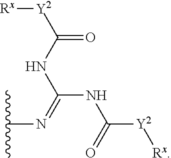

8. The compound of claim 1, wherein Z² is

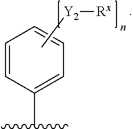

9. The compound of claim 1, wherein Z² is

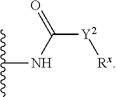

10. The compound of claim 1, wherein Y² is O.

11. The compound of claim 1, wherein R^{x} is t-butoxycarbonyl.

12. A pharmaceutical composition, comprising a compound of claim 1; and a pharmaceutically acceptable carrier.

13. A method of retarding the aging of a subject, comprising administering to the subject an effective amount of a compound of claim 1 or a compound listed in Table 1.

14. The method of claim 13, wherein the compound is 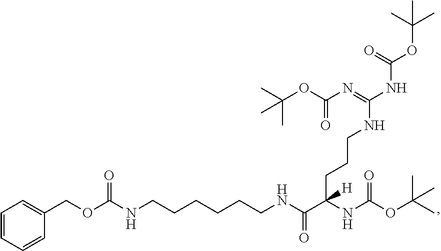 [SMILES: CC(C)(C)OC(=O)/N=C(\NCCC[C@H](NC(=O)OC(C)(C)C)C(=O)NCCCCCCNC(=O)OCc1ccccc1)NC(=O)OC(C)(C)C]
or a pharmaceutically salt thereof.

15. A method of treating cancer, treating or preventing diabetes, or treating or preventing neurodegenerative disease, comprising administering to a subject in need thereof an effective amount of a compound of claim 1 or a compound listed in Table 1.

16. The method of claim 15, wherein the compound is 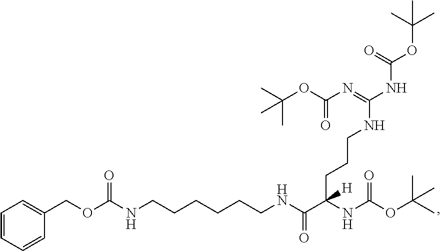 [SMILES: CC(C)(C)OC(=O)/N=C(\NCCC[C@H](NC(=O)OC(C)(C)C)C(=O)NCCCCCCNC(=O)OCc1ccccc1)NC(=O)OC(C)(C)C]
or a pharmaceutically salt thereof.

17. The method of claim 16, wherein the method is a method of treating cancer, and the method further comprises conjointly administering one or more additional chemotherapeutic agents.

18. The method of claim 15, wherein the method is a method of treating or preventing diabetes.

19. (canceled)

20. The method of claim 15, wherein the method is a method of treating or preventing neurodegenerative disease.

21. (canceled)

22. The method of claim 15, wherein the method is a method of treating cancer.
